(19) United States
(12) Patent Application Publication
(10) Pub. No.: US 2020/0224143 A1
(43) Pub. Date: Jul. 16, 2020

(54) DEVICE AND METHOD FOR CULTIVATING CELLS

(71) Applicant: Valentin Kramer, Mannheim (DE)

(72) Inventor: Valentin Kramer, Mannheim (DE)

(21) Appl. No.: 16/614,724

(22) PCT Filed: May 23, 2018
(86) PCT No.: PCT/EP2018/063470

§ 371 Date: Mar. 5, 2020

(30) Foreign Application Priority Data
May 23, 2017 (DE) 10 2017 208 758.7

(51) Int. Cl. C12M 1/00; C12M 1/06

(52) U.S. Cl. CPC C12M 29/00; C12M 23/20; C12M 27/02

(57) ABSTRACT

The invention relates to an apparatus for the culture of cells, comprising a mixing apparatus and a reaction space which is bounded by a reactor wall, wherein the reactor wall has at least one connection for the introduction of gas and/or liquid and the reaction space has an internal volume, wherein the reaction space is configured so that the internal volume can be increased by at least 500%, based on a minimum internal initial volume, or that a horizontal cross-sectional area of the reaction space increases from the bottom upward. The invention further relates to a process for the culture of cells.

The invention relates to an apparatus for the culture of cells, which comprises a mixing apparatus and a reaction space which is bounded by a reactor wall, wherein the reactor wall has at least one connection for the introduction of gas and/or liquid and the reaction space has an internal volume. The invention further relates to a process for the culture of cells in the apparatus.

Bioreactors for the culture of cells frequently comprise vessels made of glass, stainless steel or polymer which have various but fixed sizes as reaction space. Vessels made of glass or stainless steel are usually employed as multiple-use reactors and after use are cleaned, sterilized and reused. Reaction vessels made of polymer, in particular, can be intended for single use.

Furthermore, bioreactors which are made of polymer and have the form of a bag and during use are usually arranged on shakers are known. They are also referred to as wave bioreactors.

Conventional reaction vessels, in particular for the culture of cells, provide a predetermined, fixed reaction volume which is determined by an inflexible size of the vessels and generally also by an inflexible shape of the vessels.

In order to initially inoculate a bioreactor having a reaction volume of a number of liters and in particular on an industrial scale with a reaction volume of more than 1 m³ with cells it is firstly necessary to produce an amount of cells sufficient for the large reaction volume from a small amount of stored cells of a starter culture on the milliliter scale. For this purpose, cells are transferred stepwise in a seed train from a small vessel into a next-sized vessel in which cell growth is continued to the next transfer into a next-sized vessel.

In this series of precultures, the cells are optimally all in an exponential growth phase. The transfer of the cells into larger vessels is also referred to as passaging or transport. The transfer of the cells into larger volumes is necessary since many cells require a particular cell density in order to go into the exponential growth phase, also referred to as logarithmic growth phase or log phase. The cell density is usually defined as number of cells per unit volume of liquid cell culture. On reaching a maximum cell density, cell growth, namely the increase in the number of cells per unit time, frequently decreases. Dilution or addition of further medium is possible only to a limited extent because of the fixed volume of the vessels.

Cell growth in the seed train is also described as expansion of the cells. The objective of the expansion in the seed train is to provide an inoculum, which is also referred to as inoculate, which can serve for inoculation of a bioreactor on an industrial scale. For the purposes of the present invention, cell expansion, which is also described as cell line expansion, is thus the stepwise increase in the culture volume and the reproduction of the cells, usually over a plurality of process steps.

The cells are frequently present in a cell suspension in which the cells and their extracellular metabolites are suspended in a usually aqueous nutrient medium.

M. Howaldt et al., in “Kultivierung von Saugetierzellen”, Bioprozesstechnik, 3^{rd} edition, Spektrum Akademischer Verlag, 2011, pages 410 to 413, describe the industrial use of mammalian cell technology, in which the upstream processing consists of the key regions cell bank, inoculum and fermentation. To carry out fermentation, an aliquot from the cell bank is thawed and cultivated in a shaking flask or in a spinner. The cells are then transferred into next-sized culture vessels such as spinners, wave bioreactors or stainless steel bioreactors which serve as intermediates for cell reproduction production. High cell counts should be attained as quickly as possible by means of the initial culture fermenters so that the production fermenter can be inoculated with a high seed density after a very short time.

EP 2 543 719 A1 describes a meander bioreactor and a process for dynamic expansion, differentiation and harvesting of hematopoietic cells. The bioreactor consists of a bioreactor vessel whose bottom is provided with offset dividing walls which bring about meandering passage of media with laminar flow. A plurality of meander bioreactors can be connected in series in a module-like manner via shut-off valves. Cells introduced form a cell layer which virtually covers the bottom area.

In “Considerations for Cell Passaging in Cell Culture Seed Trains”, BMC Proceedings, 2015, 9 (Suppl 9): P43, T. H. Rodriguez et al. describe a software-based tool for optimizing seed trains, in which an optimum point in time for transferring the cell culture from one scale into the next-sized scale is calculated.

In “A Novel Seed-Train Process”, BioProcess International 13(3)s, March 2015, pages 16 to 25, B. Wright et al. compare an improved seed train with a conventional seed train, in which the required time and the complexity of the improved seed train are reduced by use of a high-density cell culture.

In the guidelines for the handling of culture bags by the manufacturer Miltenyi Biotec GmbH, Handling guidelines MACS® GMP Cell Culture Bags, September 2013, a description is given of culture bags for cell expansion which are divided into three chambers which can be connected to one another by opening welded seams to give a volume of 100 ml.

The stepwise transfer of the cells into next-sized vessels in the expansion of the cells represents a time-consuming and laborious working step. During the transfer, the cells are also temporarily located outside the culture vessels, so that there is an increased risk of undesirable contamination of the cell culture.

It is an object of the present invention to provide an apparatus and a process by means of which the transfer of the cells into a plurality of vessels of different sizes during cell expansion is avoided.

The object is achieved by an apparatus for the culture of cells, comprising a mixing apparatus and a reaction space which is bounded by a reactor wall, wherein the reactor wall has at least one connection for the introduction of gas and/or liquid and the reaction space has an internal volume, wherein the reaction space is configured so that the internal volume can be increased by at least 500%, preferably by at least 700%, more preferably by at least 1000%, even more preferably by at least 1500% and particularly preferably by at least 2000%, based on a minimum internal initial volume, or that a horizontal cross-sectional area of the reaction space increases from the bottom upward.

Preference is given to the internal volume not being able to be enlarged by more than 10 000%, based on a minimum internal initial volume.

The object is also achieved by a process for the culture of cells in the apparatus of the invention, comprising the following steps:
a. optionally sterilization of the reactor wall at a temperature of from 80° C. to 150° C. or by means of gamma radiation,
b. charging of the apparatus, with cells and optionally a first liquid medium being introduced into the reaction space,
c. metering of a second liquid medium and/or a gas into the reaction space, with the second liquid medium preferably having a composition which corresponds to that of the first liquid medium, and
   enlargement of the internal volume of the reaction space by at least 500%, preferably by at least 700% and more preferably by at least 1000%, based on the minimum internal initial volume, or
   enlargement of a fill height in the reaction space, whose horizontal cross-sectional area increases from the bottom upward, with a volume of a liquid phase in the reaction space, which comprises the second liquid medium and optionally the first liquid medium, increasing by at least 500%, based on a minimum liquid initial volume.

Due to the internal volume of the reaction space being able to be enlarged by at least 500%, larger amounts of liquid medium can be introduced into the same vessel, so that despite an increasing number of cells in the reaction space the cell density based on a unit volume of liquid medium does not exceed a maximum cell density and transfer of the cell culture into a larger vessel is avoided.

An internal volume of the reaction space which is variable according to the invention is also advantageous because the minimum internal initial volume is also adjustable in addition to the final volume being enlarged. This makes possible a smaller ratio of surface area to volume of the cell culture compared to the use of large vessels at the beginning when only a small amount of cell culture comprising cells and medium is present. In particular, a phase interface between the cell culture, i.e. the liquid phase, and a gas phase which is likewise present in the vessel is also relatively small at the beginning.

If, on the other hand, only a small amount of cell culture is present in a comparatively large vessel, the ratio of surface area to volume of the cell culture is large and evaporation of the medium through to drying out of the cells occurs. This is the case especially when the culture of the cells is carried out at a temperature above room temperature.

The volume of cell culture present at the beginning of cell expansion is also usually small because going below a minimum cell density in the cell culture can lead to decreased cell growth.

The internal volume of the reaction space according to the invention, with cell growth being considered to be a reaction here, increases essentially as a function of the number of cells present in the reaction space until a desired volume of cell culture having a desired cell density has been attained. The reaction space is enlarged during expansion of the cell culture and optionally changes its shape.

Avoidance of the transfer of the cells from a relatively small vessel into a larger vessel reduces the risk of undesirable contamination. The amount of work involved is likewise decreased, as is the outlay for material and cleaning of further vessels.

The internal volume is preferably a continuous volume. For the purposes of the invention, a continuous volume means that there is no division into a plurality of sections which, for example, are connected to one another only by pipe or tube connections.

For the purposes of the invention, the minimum internal initial volume is a smallest internal volume of the reaction space which can be set. The minimum internal initial volume is preferably in the range from 2 ml to 1 1, more preferably in the range from 5 ml to 500 ml and particularly preferably in the range from 5 ml to 100 ml. The minimum internal initial volume can be increased to a maximum internal volume of the reaction vessel and the maximum internal volume is preferably in the range from 101 to 100 l, particularly preferably in the range from 15 l to 50 l.

The internal volume is preferably capable of being enlarged continuously. For the purposes of the invention, capable of being enlarged continuously means that the internal volume increases uniformly over time and/or that every degree of increase of the internal volume up to the maximum internal volume can be set steplessly.

The reactor wall advantageously comprises a wall material which is biocompatible and not toxic to cell cultures. The wall material is preferably impermeable to liquids and stable to sterilization by means of heat, radiation and/or chemicals.

The wall material is preferably thermally stable in the range from 10° C. to 150° C., more preferably in the range from 25° C. to 130° C. In addition, the wall material is preferably stable in the presence of liquids having a pH in the range from pH 5 to pH 8, more preferably in the range from pH 6 to pH 7. The wall material also preferably displays a low swelling behavior in water and is chemically and physically resistant to water and aqueous solutions.

Furthermore, the wall material preferably has a density in the range from 0.7 to 1.6 g/cm³, more preferably in the range from 0.9 to 1.6 g/cm³ and particularly preferably in the range from 0.9 to 1.1 g/cm³.

As wall material, the reactor wall preferably comprises an extensible material, where the extensible material has an extensibility of at least 200% and/or the reactor wall has a first part and a second part, where the first part is arranged movably and with a sealing connection relative to the second part.

Preference is given to the extensible material being extended in step c) and/or the first part of the reactor wall being moved relative to the second part of the reactor wall in step c).

An extension ε is usually considered to be a relative change in length of a body under load, in particular the ratio of a length change Δl to an original length l₀:

$$ɛ = \frac{Δ l}{{l}_{0}}$$

The extensible material is preferably extensible in at least two directions in space, with the reported extensibility being based on one of the two directions in space.

The extensibility describes the ability of the extensible material to change its shape under the action of force and indicates how far the material can be extended without breaking or tearing. The extensibility, which is also referred to as ultimate tensile strength, is usually determined by means of a tensile test in accordance with DIN 53504:2017-03.

Hook's law describes the dependence of the extension ε on a stress σ, where E is the modulus of elasticity, also referred to as E modulus:

σ=E·ε

The extensible material preferably has an E modulus, which can be determined in accordance with DIN 53504:2017-03, in the range from 0.1 to 0.5 GPa, more preferably from 0.02 to 0.1 GPa and particularly preferably from 0.03 to 0.07 GPa. Furthermore, the Shore A hardness of the extensible material, which can be determined in accordance with DIN ISO 7619-1:2012-02, is preferably in the range from 20 to 100, more preferably from 30 to 80.

The extensible material preferably has a high elasticity. A material is described as elastic when after releasing of the load, it returns to the undeformed initial state which was present before application of the load.

The extensible material also preferably has an ultimate tensile strength in the range from 3.5 to 41.4 MPa, more preferably from 5.5 to 35.9 MPa. The extensible material more preferably has an extensibility of at least 300%, more preferably at least 330% and particularly preferably at least 500%. The extensible material usually has an extensibility of up to 5000%, preferably up to 2000% and more preferably up to 1500%. A measurement method for determining ultimate tensile strength and extensibility is described in DIN 53504:2017-03.

The extensible material preferably comprises at least one elastomer, in particular a thermoplastic elastomer. Furthermore, the extensible material preferably contains a polymer selected from the group consisting of synthetic rubber such as styrene-butadiene rubber, chloroprene rubber, polybutadiene rubber, ethylene-propylene-diene rubber (EPDM), silicone rubber, fluoro rubber, nitrile rubber, polyurethane (PU), hydrogenated acrylonitrile-butadiene rubber (HNBR), polypropylene, polyisobutylene and polyisopropene (PI), natural rubber such as latex and mixtures thereof.

Particular preference is given to the extensible material containing polyisopropene, polybutylene and/or silicone. The extensible material preferably contains more than 50% by weight, based on the extensible material, particularly preferably more than 80% by weight and in particular more than 90% by weight, of polyisopropene, polybutylene and/or silicone. The extensible material preferably contains up to 100% by weight, based on the extensible material, of polyisopropene, polybutylene and/or silicone and the extensible material particularly preferably consists of polyisopropene, polybutylene and/or silicone.

The extensible material can also contain additives such as proteins, in particular casein and/or collagens. The extensible material preferably contains plasticizers such as carboxylic esters, fats, oils and camphor.

Natural and synthetic rubbers are particularly suitable since they have a high elasticity. Polyurethane has the advantage of a high melting point.

Preference is given to an area of the reactor wall which faces the reaction space being able to be enlarged. The area is preferably enlarged in step c) of the process of the invention. To effect the enlargement of the area facing the reaction space, the extensible material is advantageously extended. The extensible material is preferably extended by metering the second liquid medium and/or the gas into the reaction space. The metered introduction of the second liquid medium and/or the gas is preferably carried out by means of transport under superatmospheric pressure.

As an alternative or in addition, the first part of the reactor wall is moved relative to the second part of the reactor wall in such a way that an area of the second part of the reactor wall which faces the reaction space and bounds the reaction space becomes greater. Here, a section of the second part of the reactor wall is initially not in direct contact with the reaction space and only after moving of the first part of the reactor wall does it face the reaction space directly and bound the reaction space. The first part and the second part are arranged in such a way that a sealing but movable connection is present between the first part and the second part of the reactor wall. The sealing connection preferably comprises a rubber seal or a rubber lip.

In one embodiment, the reactor wall advantageously has the shape of a lateral surface of a cylinder, which can also be referred to as tube. One side of the lateral surface is preferably closed in a fixed manner by a bottom area. The lateral surface and optionally the bottom area preferably represent the second part of the reactor wall. The first part of the reactor wall preferably comprises a second bottom area which can also be referred to as lid and is arranged in a movable manner on the other side of the lateral surface, more preferably within the lateral surface.

In a further embodiment, the reaction space is configured so that a horizontal cross-sectional area of the reaction space increases from the bottom upward, with the direction from the bottom upward being opposite to the direction of gravity. The horizontal cross-sectional area of the reaction space preferably increases continuously, and a first horizontal cross-sectional area of the reaction space is more preferably at least twice the size of a second horizontal cross-sectional area of the reaction space at a second height, the first horizontal cross-sectional area is located above the second horizontal cross-sectional area and there is particularly preferably a distance between the first horizontal cross-sectional area and the second horizontal cross-sectional area which corresponds to less than 50% of a maximum vertical dimension of the reaction space. During the course of the process of the invention, the fill level of the liquid phase in the reaction space increases, so that the fill height in the reaction space whose horizontal cross-sectional area increases from the bottom upward increases and the volume of the liquid phase in the reaction space, which comprises the second liquid medium and optionally the first liquid medium, increases by at least 500%, more preferably by at least 1000%, and preferably by not more than 10 000%, based on a minimum liquid initial volume. The minimum liquid initial volume is the volume of the liquid phase in the reaction space before the second liquid medium is metered in in step c). The minimum liquid initial volume is preferably in the range from 1.5 ml to 800 ml, more preferably in the range from 3 ml to 400 ml and particularly preferably in the range from 4 ml to 100 ml. The fill height is based on the liquid phase and is the longest vertical distance of the liquid surface facing the gas phase from the reactor wall or a reactor base. The ratio of the phase interface between the liquid phase and the gaseous phase in the reaction space to the volume of the liquid phases preferably changes by less than a factor of 10, more preferably by less than a factor of 5, during the metered introduction of the second liquid medium. The reaction space is optionally closed by means of a lid, which preferably comprises a flexible material. The at least one connection for the introduction of gas and/or liquid can also be arranged on the lid.

The reactor wall preferably has a conical shape, with the tip of the cone pointing downward, i.e. in the direction of gravity.

As an alternative or in addition, the reactor wall can be configured at least partly as a bellows, with the reactor wall being at least partly folded in an accordion-like manner.

The apparatus and in particular the reactor wall can be configured for a single use or for multiple uses. In the case of a single use, the reaction space can also be referred to as single-use reactor. In this case, the reactor wall is, in particular, disposed of after one use for the culture of cells. In the case of optionally multiple uses, at least the reactor wall and the at least one connection for the introduction of gas and/or liquid, preferably all surfaces of the apparatus for the culture of cells which come into contact with cells, are cleaned and sterilized after the culture of the cells has been completed and are reused.

In step b) of the process of the invention, the cells and optionally a first liquid medium are initially placed in the reaction space. As an alternative, cells can also firstly be placed in the reaction space and are then suspended in the second liquid medium, preferably in step c) of the process of the invention, or the first liquid medium can be placed in the reaction space first, followed by addition of the cells thereto.

Preference is given to the first liquid medium and the second liquid medium having the same composition. A composition which is the same also encompasses the case where a content of extracellular metabolites of the cells is different in the first liquid medium and the second liquid medium. Thus, the cells can be initially charged in a suspension in the first liquid medium which is then merely diluted by addition of the second liquid medium, so that the cell suspension before and after step c) essentially differs only in terms of the cell density but not in terms of the composition of the medium.

In an alternative embodiment, the composition of the first liquid medium can differ from the composition of the second liquid medium. Thus, for example, the composition of the second liquid medium can be matched to the progressed growth phase of the cells.

The second liquid medium and optionally the first liquid medium preferably contain at least 50% by weight of water, more preferably at least 75% by weight and particularly preferably at least 90% by weight of water. The second liquid medium and optionally the first liquid medium preferably contain up to 99% by weight of water.

Furthermore, the second liquid medium and optionally the first liquid medium preferably contain inorganic salts such as CaCl₂, Fe(NO₃)₃, KCl, MgSO₄, NaCl, NaH₂PO₄ and/or NaHCO₃, amino acids such as L-arginine, L-cystine, L-glutamine, glycine, L-histidine, L-isoleucine, L-leucine, L-lysine, L-methionine, L-phenylalanine, L-serine, L-threonine, L-tryptophan, L-thyrosine and/or L-valine, vitamins such as D-calcium pantothenate, choline chloride, folic acid, i-inositol, niacinamide, riboflavin and/or thiamine, and/or other components such as D-glucose, phenol red and/or sodium pyruvate.

The second liquid medium and optionally the first liquid medium particularly preferably contain at least 50% by weight of Dulbecco's Modified Eagle's Medium (DMEM), more preferably at least 75% by weight and particularly preferably at least 90% by weight of DMEM.

The second liquid medium is preferably introduced continuously. As an alternative, the second liquid medium can be introduced stepwise, which can also be referred to as in portions. The rate of introduction in the case of continuous introduction can be constant or can be matched to cell growth. In the case of stepwise introduction, the point in time and the volume added per step can be matched to cell growth or the introduction can occur after a fixed time interval and with a fixed volume.

Owing to the introduction of the second liquid medium, the process of the invention can also be referred to as fed-batch process.

Furthermore, further gas and/or further liquid can additionally be introduced into the reaction space. The reactor wall preferably has at least two connections for the introduction of gas and/or liquid. The metered introduction of the second liquid medium and/or the gas in step c) is advantageously effected through the connections, and the second liquid medium and/or the gas are particularly preferably each introduced separately through a connection. In particular, the reactor wall can comprise connections for introduced air and/or oxygen, carbon dioxide, nitrogen and/or exhaust air from the reaction space. The apparatus for the culture of cells preferably comprises connections which have sterile filters. Preference is given to the reaction space being continuously supplied with gas and gas being continuously discharged from the reaction space, so that continuous replacement of the gas phase in the reaction space takes place.

It is also possible for connections, preferably without sterile filters, which connect the reaction space with sterile stock vessels to be present, so that there are connections for introduction of further liquids, in particular nutrient medium. A connection for the taking of samples can also be provided.

The reactor wall preferably has a connecting piece on which the at least one connection for the introduction of gas and/or liquid is arranged.

The cells, the second liquid medium and optionally the first liquid medium are preferably mixed in the reaction space, in particular with the aid of the mixing apparatus. The mixing apparatus can comprise a mixer having a rotating shaft, a vibratory mixer, a hydraulic mixer, a pneumatic mixer, a static mixer or a shaker.

For example, the mixing apparatus is arranged in the form of a stirrer, in particular in the form of a magnetic stirrer, in the reaction space. The reaction space is preferably arranged on a shaker such as a shaking table or an orbital shaker. Furthermore, the reaction space is fastened on the shaker by means of fastening means, with the fastening means preferably being extensible. The second liquid medium and optionally the first liquid medium containing the cells is preferably set into wave motion by means of the mixing apparatus.

The reaction space preferably contains the gaseous phase and the liquid phase, where the liquid phase, which is preferably a suspension, comprises the second liquid medium, the cells and optionally the first liquid medium. The volume ratio of the gaseous phase to the liquid phase in the reaction space preferably remains essentially unchanged, which can be brought about by the enlargement of the internal volume during metered introduction of the second liquid medium and/or the gas. In this way, the size of the phase interface between the liquid phase and the gaseous phase relative to the volume of the liquid phase also remains essentially constant, so that evaporation effects also remain essentially unchanged over the culture time.

Before the metered introduction in step c), the liquid phase preferably occupies from 20% by volume to 80% by volume, more preferably from 30% by volume to 70% by volume and particularly preferably from 40% by volume to 60% by volume, of the internal volume.

Furthermore, after the metered introduction in step c), the liquid phase preferably occupies from 10% by volume to 80% by volume, more preferably from 20% by volume to 60% by volume and particularly preferably from 30% by volume to 50% by volume, of the internal volume. The presence of the gaseous phase in the reaction space, which preferably contains oxygen, is particularly advantageous in the culture of aerobic cells.

The cells are preferably present in a suspension culture in the reaction space. The cells comprise, in particular, nonadhesive cells, which is intended to mean that the cells essentially do not adhere to the reactor wall or other surfaces in the reaction space.

The cells preferably comprise eukaryotic cells which are present in a suspension culture, in particular mammalian cells, plant cells, insect cells and/or fungus cells, particularly preferably mammalian cells. As an alternative or in addition, the cells can also comprise prokaryotic cells such as bacteria.

It is likewise conceivable to use the apparatus for the cultivation of adherent cells, so that the cells present in the reaction space comprise adherent cells.

In step c) of the process of the invention, the number of cells present in the reaction space preferably increases by cell growth. For this purpose, the reaction space is preferably maintained at a temperature in the range from 15° C. to 50° C., more preferably from 20° C. to 45° C., even more preferably from 35° C. to 40° C. and particularly preferably from 36° C. to 48° C.

### BRIEF DESCRIPTION OF THE DRAWINGS

A working example according to the prior art and embodiments according to the invention are shown in the drawings and are explained in more detail in the following description.

The drawings show:

FIG. 1 a schematic depiction of a conventional seed train;

FIG. 2 an apparatus according to the invention for the culture of cells, having a reactor wall comprising an extensible material;

FIG. 3 an apparatus according to the invention for the culture of cells, having a reactor wall comprising a movable first part and a second part,

FIG. 4 an apparatus according to the invention for the culture of cells, having a conical reactor wall,

FIG. 5 a side view of a reaction space having a reactor wall comprising an extensible material,

FIG. 6 a further view of the reaction space having a reactor wall comprising an extensible material,

FIG. 7 a schematic depiction of an apparatus for the culture of cells, having a reaction space with a reactor wall made of an extensible material.

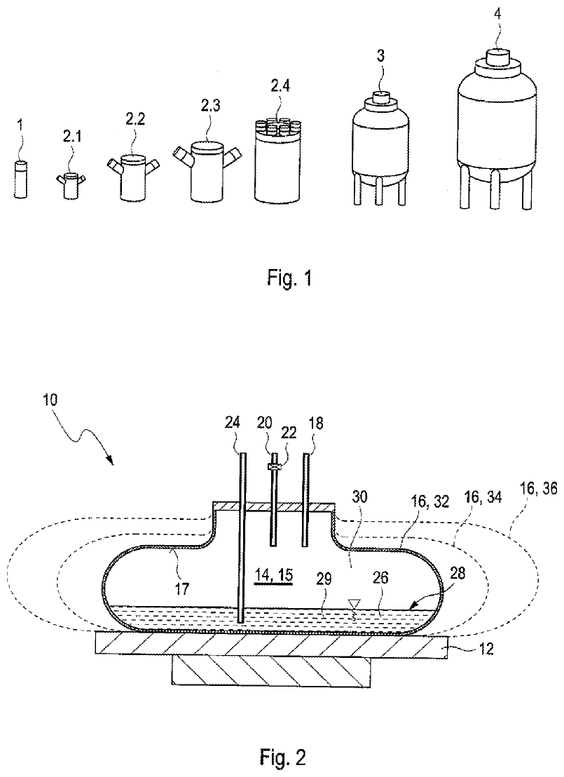

FIG. 1 schematically depicts the course of a conventional seed train. From a storage vessel 1, which contains a starter culture, a cell culture in the form of a cell suspension comprising cells and a culture medium is introduced in a volume of 1 ml and with a cell density of 2×10⁷ cells/ml into a first expansion vessel 2.1. Overall, four expansion vessels 2.1, 2.2, 2.3 and 2.4 are used in order to be able to produce a sufficient number of cells as inoculum for inoculating a production vessel 4. Since a maximum volume-specific cell density should not be exceeded so as not to hinder cell growth, fresh medium is added to the cell suspension with increasing number of cells during the expansion.

When a maximum fill level has been reached in the first expansion vessel 2.1, the cell suspension is transferred from the first expansion vessel 2.1 into the second expansion vessel 2.2 and further fresh medium is added in an amount corresponding to cell growth. The cell suspension is subsequently transferred accordingly into the third expansion vessel 2.3 and then into the fourth expansion vessel 2.4.

From the first expansion vessel 2.1 to the fourth expansion vessel 2.4, the volume of the cell suspension is increased from 125 ml to 10 l. A preproduction vessel 3, which has an internal volume of 50 l and is also referred to as stage n−1, is inoculated with the cell suspension present in the fourth expansion vessel 2.4. In the preproduction vessel 3, the number of cells continues to increase so that the contents of the preproduction vessel 3 serve to inoculate the production vessel 4 which has an internal volume of 500 l. The vessels shown here are batch reactors.

FIG. 2 shows a schematic depiction of an apparatus according to the invention for the culture of cells 10, having a reactor wall 16 comprising an extensible material. An apparatus for the culture of cells 10, which can replace the four expansion vessels 2.1, 2.2, 2.3 and 2.4 of FIG. 1 since the required volume increase of the cell suspension can be effected in the same vessel, is depicted.

The apparatus for the culture of cells 10 comprises a mixing apparatus 12 on which a reaction space 14 is arranged. The mixing apparatus 12 is, for example, an orbital shaker. The reaction space 14 having an internal volume 15 is bounded by a reactor wall 16. The reactor wall 16 comprises a connection for the introduction of liquid 18 and a connection for the introduction of gas 20, with the connection for the introduction of gas 20 having a sterile filter 22. In addition, a tube 24 for taking samples is provided.

The reaction space 14 contains both a liquid phase 28 and a gaseous phase 30. The liquid phase 28 contains a second liquid medium 26 and optionally a first liquid medium and also cells 29.

In an initial state 32, the reaction space 14 has a minimum internal initial volume and the reactor wall 16, which in this embodiment comprises an extensible material, is extended by introduction of a second liquid medium 28 and/or gas through the connection for introduction of liquid 18 and/or through the connection for introduction of gas 20, so that a first state of extension 34 is attained. An area 17 of the reactor wall 16 facing the reaction space is enlarged by extension of the extensible material. The reactor wall 16 is extended further by further introduction of second liquid medium 26 or gas, so that a second state of extension 36 is attained.

In contrast to the vessels 2.1, 2.2, 2.3 and 2.4 of FIG. 1, in the embodiment according to the invention as shown in FIG. 2 not only does the volume of the liquid phase 28, i.e. the cell suspension, increase but the internal volume 15 of the reaction space 14 also increases, so that transfer of the liquid phase 28 into a next-sized vessel is unnecessary until a maximum internal volume of the reaction space 14 according to the invention has been reached. Accordingly, cell expansion can be carried out in only one vessel.

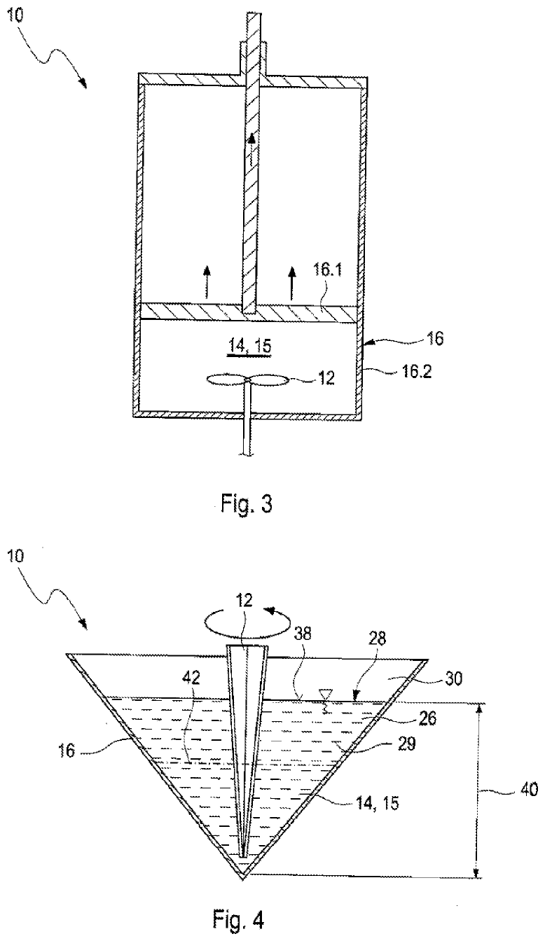

FIG. 3 shows an apparatus according to the invention for the culture of cells 10, having a reactor wall 16 comprising a movable first part 16.1 and a second part 16.2. The apparatus according to the invention for the culture of cells 10 comprises a mixing apparatus 12, with the mixing apparatus 12 being a stirrer in this embodiment.

The reaction space 14 having the internal volume 15 is bounded by the first part 16.1 of the reactor wall 16 and the second part 16.2 of the reactor wall 16. The first part 16.1 is arranged movably and in sealing connection with the second part 16.2. The second part 16.2 has the shape of a tube or a cylindrical surface and the first part 16.1 of the reactor wall 16 can be moved upward inside the second part 16.2, so that the internal volume 15 of the reaction space 14 is enlarged.

FIG. 4 shows an apparatus according to the invention for the culture of cells 10, having a conical reactor wall 16. The apparatus for the culture of cells 10 comprises a mixing apparatus 12 in the form of a stirrer. The reaction space 14 having an internal volume 15 has a conical shape.

Both a liquid phase 28 comprising the second liquid medium 26, optionally the first liquid medium and cells 29, and also a gaseous phase 30 are present in the reaction space 14 whose horizontal cross-sectional area 42 increases from the bottom upward. The liquid phase 28 reaches a fill height 40. The conical shape makes it possible, in contrast to reactor walls 16 having different shapes, for there to be a sufficiently small ratio of the phase interface 38 between the liquid phase 28 and the gaseous phase 30 to the volume of the liquid phase 28 even at small volumes of the liquid phase 28, so that evaporation effects do not prevail. Nevertheless, a required increase in the volume of the liquid phase 28 during cell expansion is possible.

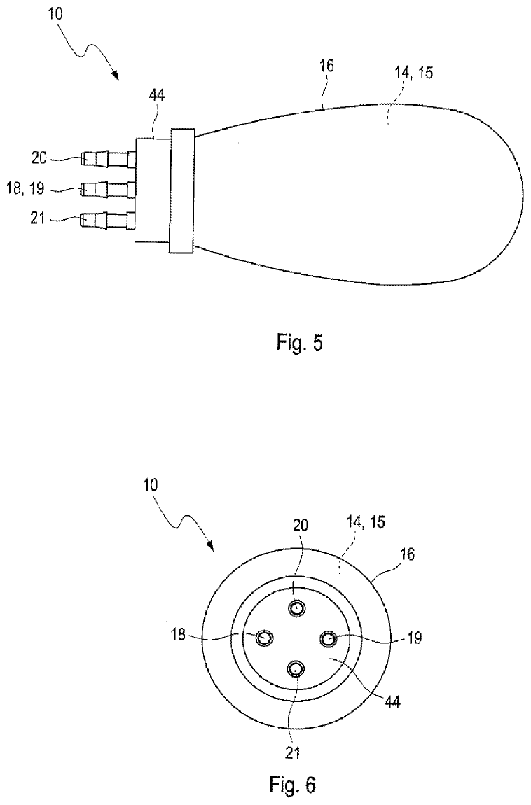

FIG. 5 shows a side view of a reaction space 14 having a reactor wall 16 comprising an extensible material. The reaction space 14 has an internal volume 15 which can be enlarged by extension of the extensible material. The reactor wall 16 has a connecting piece 44 on which a connection for the introduction of liquid 18, a connection for the taking of samples 19, a connection for the introduction of gas 20 and a gas outlet 21 are arranged.

FIG. 6 shows a further view of the reaction space 14 having the reactor wall 16 comprising an extensible material as per FIG. 5. The connecting piece 44 has a connection for the introduction of liquid 18, a connection for the taking of samples 19, a connection for the introduction of gas 20 and a gas outlet 21.

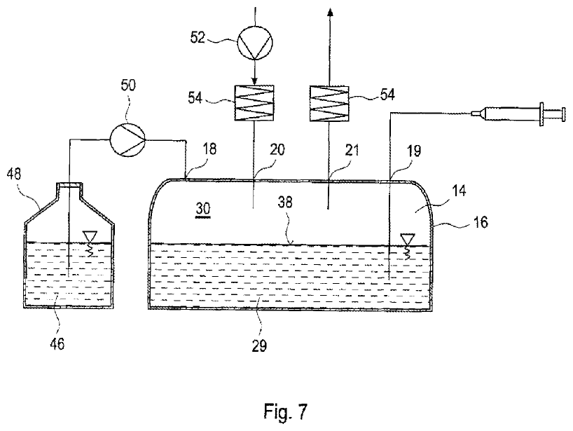

FIG. 7 schematically depicts an apparatus for the culture of cells, having a reaction space made of an extensible material.

The reaction space 14 made of extensible material can, for example, have a construction as depicted in FIGS. 5 and 6. However, a preferred alternative is for the reaction space 14 to be completely enclosed by a reactor wall 16 made of extensible material and the connection for the introduction of liquid 18, the connection for the introduction of gas 20, the gas outlet 21 and the connection for the taking of samples 19 to be conducted directly through the extensible material forming the reactor wall. The sealing of the connection for the introduction of liquid 18, the connection for the introduction of gas 20, the gas outlet 21 and the connection for the taking of samples 19 is effected here in a customary manner known to a person skilled in the art, for example by adhesive bonding.

For the culture of cells, culture medium 46 is transported from a stock vessel 48 by means of a pump 50, preferably a pneumatic pump, via the connection for the introduction of liquid 18 into the reaction space. The pumping capacity of the pump is dependent on the growth of the cells 29 which are cultured in the reaction space 14. Here, the culture medium 46 can be conveyed either continuously or stepwise into the reaction space 14, with further culture medium being pumped in when that present in the reaction space has virtually been consumed. As an alternative to a pneumatic pump, it is also possible to use any other pump by means of which contamination-free transport of the culture medium 46 into the reaction space 14 is possible.

The gas necessary for the culture of the cells, usually an oxygen-containing gas such as pure oxygen or air or else oxygen-enriched air, is introduced via the connection for the introduction of gas 20 into the reaction space. The introduction of the gas is carried out by means of a gas pump 52. In order to prevent contamination being introduced together with the gas into the reaction space, the gas is passed through a sterile filter 54 before introduction into the reaction space. A membrane pump, for example, is suitable as gas pump 52. However, it is also possible here to use any other suitable pump by means of which the gas can be conveyed.

Excess gas can be taken off through the gas outlet 21 from the reaction space 14. In order to prevent undesirable components being discharged together with the gas from the reaction space 14, the gas outlet 21 is likewise adjoined by a sterile filter 54.

The connection for the taking of samples 19 is connected to a suitable sampling system 56. Here too, in order for no contamination to be able to be introduced into the system and the samples taken also not to be contaminated, it is necessary for the sampling system 56 to be sterile. In order to take a sample from the reaction space 14, it is necessary for a pressure which is below the pressure in the reaction space 14 to be able to be applied at the connection for the taking of samples 19, so that the sample is taken from the reaction space 14 due to the pressure difference. A suitable sampling system is, for example, a syringe, the syringe piston of which is pulled back for sampling, so that the space for accommodating the sample is enlarged during sampling.

### EXAMPLE 1

A liquid phase having a volume of 10 ml was placed in an apparatus for the culture of cells as per FIG. 2, which had an extensible material as wall material. Over ten hours, an aqueous medium and air were continuously introduced until the liquid phase in the reaction space had reached a volume of 20 l.

The experiment was carried out using three different wall materials. The reactor wall consisted essentially of natural rubber, polyisoprene or polyurethane, respectively, which each have good biocompatibility. The reactor wall had a wall thickness of about 100 μm at the beginning before extension.

In the case of all three materials, a volume increase of the internal volume of at least 2000% was attained. The reactor wall made of polyisoprene and the reactor wall made of natural rubber had an even greater extensibility than the reactor wall made of polyurethane.

### EXAMPLE 2

500 ml of an aqueous suspension containing yeast cells (Saccharomyces cerevisiae) were placed in an apparatus for the culture of cells as per FIG. 2, which had a reactor wall consisting essentially of polyisoprene. The cells were cultured over a period of 14 days, during which a total additional amount of 4.5 l of aqueous medium was added stepwise. The production of metabolites could be observed over the total culture time.

### EXAMPLE 3

An apparatus for the culture of cells as per FIG. 2, which had a reactor wall consisting essentially of polyisoprene, was autoclaved at 120° C. and 2 bar for 2 hours. 50 ml of cell culture medium consisting of Dulbecco's Modified Eagle's Medium (DMEM), containing 10% by weight of fetal calf serum (FCS) were incubated in the autoclaved apparatus at 37° C. for 7 days, and a sample of the cell culture medium was then examined under a microscope. At an enlargement of 10 000×, no cells were observed. The apparatus was consequently suitable for sterile work.

### LIST OF REFERENCE NUMERALS

1 Storage vessel
2 Expansion vessels
2.1 First expansion vessel
2.2 Second expansion vessel
2.3 Third expansion vessel
2.4 Fourth expansion vessel
3 Preproduction vessel
4 Production vessel
10 Apparatus for the culture of cells
12 Mixing apparatus
14 Reaction space
15 Internal volume
15 16 Reactor wall
16.1 First part
16.2 Second part
17 Area facing the reaction space
18 Connection for the introduction of liquid
19 Connection for the taking of samples
20 Connection for the introduction of gas
21 Gas outlet
22 Sterile filter
24 Tube
26 Second liquid medium
28 Liquid phase
29 Cells
30 Gaseous phase
32 Initial state
34 First state of extension
36 Second state of extension
38 Phase interface
42 Horizontal cross-sectional area
44 Connecting piece
46 Culture medium
48 Stock vessel
50 Pump
52 Gas pump
54 Sterile filter
56 Sampling system

1. An apparatus for the culture of cells (10), comprising a mixing apparatus (12) and a reaction space (14) which is bounded by a reactor wall (16), wherein the reactor wall (16) has at least one connection (18, 20) for the introduction of gas and/or liquid and the reaction space (14) has an internal volume (15), wherein the reaction space (14) is configured so that the internal volume (15) can be increased by at least 500%, based on a minimum internal initial volume, or that a horizontal cross-sectional area (42) of the reaction space (14) increases from the bottom upward.

2. The apparatus as claimed in claim 1, characterized in that the reactor wall (16) comprises an extensible material, where the extensible material has an extensibility of at least 200% and/or the reactor wall (16) has a first part (16.1) and a second part (16.2), where the first part (16.1) is arranged movably and with a sealing connection relative to the second part (16.2).

3. The apparatus as claimed in claim 2, characterized in that the extensible material contains natural rubber, synthetic rubber, polyisobutylene, silicone rubber or mixtures thereof.

4. The apparatus as claimed in any of claims 1 to 3, characterized in that the minimum internal initial volume is in the range from 2 ml to 1 l and can be enlarged to a maximum internal volume of from 10 l to 100 l.

5. The apparatus as claimed in any of claims 2 to 4, characterized in that the at least one connection (18, 20) for the introduction of gas and/or liquid is conducted through the extensible material of the reactor wall (16).

6. A process for the culture of cells in an apparatus as claimed in any of claims 1 to 5, comprising the following steps:
a. optionally sterilization of the reactor wall (16) at a temperature of from 80° C. to 150° C. or by means of gamma radiation,
b. charging of the apparatus, with cells (29) and optionally a first liquid medium being introduced into the reaction space (16),
c. metering of a second liquid medium (26) and/or a gas into the reaction space (14), with the second liquid medium preferably having a composition which corresponds to that of the first liquid medium (26), and
enlargement of the internal volume (15) of the reaction space (14) by at least 500%, based on the minimum internal initial volume, or
enlargement of a fill height in the reaction space (14), whose horizontal cross-sectional area (42) increases from the bottom upward, with a volume of a liquid phase (28) in the reaction space (14), which comprises the second liquid medium (26) and optionally the first liquid medium, increasing by at least 500%, based on a minimum liquid initial volume.

7. The process as claimed in claim 6, characterized in that the reactor wall (16) comprises an extensible material and the extensible material is extended in step c) and/or the reactor wall (16) has a first part (16.1) and a second part (16.2) and the first part is moved relative to the second part (16.1) in step c).

8. The process as claimed in claim 6 or 7, characterized in that the second liquid medium (26) is introduced continuously and/or stepwise.

9. The process as claimed in any of claims 6 to 8, characterized in that the cells (29), the second liquid medium (26) and optionally the first liquid medium are mixed in the reaction space (14).

10. The process as claimed in any of claims 6 to 9, characterized in that the liquid phase (28) occupies from 10% by volume to 80% by volume of the internal volume (15) before and after the metered introduction in step c).

11. The process as claimed in any of claims 6 to 10, characterized in that the cells (29) are present in a suspension culture in the reaction space (14).
